(19) United States
(12) Patent Application Publication
(10) Pub. No.: US 2017/0056075 A1
(43) Pub. Date: Mar. 2, 2017

(54) A SPINAL PROBE INCORPORATING AN ELECTROMECHANICAL SYSTEM FOR DETECTION AND PREVENTION OF BREACHES DURING SURGERY

(71) Applicant: THE JOHNS HOPKINS UNIVERSITY, Baltimore, MD (US)

(72) Inventors: Anvesh Annadanam, Denville, NJ (US); Robert Allen, Baltimore, MD (US); Chetan Bettegowda, Perry Hall, MD (US); Ravi Gaddipati, Colorado Springs, CO (US); Luis Herrera, South Gate, CA (US); Bradley Isaacs, Merrick, NY (US); Sheng-fu Lo, Baltimore, MD (US); Eric Xie, Hudson, OH (US); Clay Andrews, Westport, CT (US); Adarsha Malla, Dublin, GA (US); Erica Schwarz, Sahuarita, AZ (US)

(21) Appl. No.: 15/308,885

(22) PCT Filed: May 6, 2015
(86) PCT No.: PCT/US2015/029371

§ 371 Date: Nov. 4, 2016

Related U.S. Application Data
(60) Provisional application No. 61/989,339, filed on May 6, 2014.

(51) Int. Cl. A61B 17/70; A61B 90/00; A61B 17/34

(52) U.S. Cl. CPC A61B 17/7074; A61B 17/3472; A61B 90/06; A61B 2017/00115

(57) ABSTRACT

The present invention is directed to an innovative pedicle probe that uses a force-sensing electromechanical system coupled with haptic and visual feedback. The probe of the present invention reduces the rate of pedicle screw breaches during spinal fusion surgery. The probe provides an effective guidance system to aid surgeons in detecting and preventing cortical bone breaches, thereby minimizing risk of intraoperative injury to the patient. Moreover, the probe invention decreases surgeon reliance on intraoperative radiation, reducing harmful exposure to both patients and surgeons.

### CROSS REFERENCE TO RELATED APPLICATIONS

This application claims the benefit of U.S. Provisional Patent Application No. 61/989,339 filed May 6, 2014, which is incorporated by reference herein, in its entirety.

### FIELD OF THE INVENTION

The present invention relates generally to medical devices. More particularly, the present invention relates to a probe for spinal fusion surgery.

### BACKGROUND OF THE INVENTION

Spinal fusion through screw-based stabilization is a widely-performed and increasingly prevalent surgical technique used to alleviate spinal instabilities and deformities. The purpose of this procedure is to align and reinforce the spine through the placement of screws within affected vertebrae and subsequently join these screws with a fixative element such as metal rods. Of the approximately 500,000 spinal fusion surgeries performed annually in the United States, over 20% of screws are misplaced. This potentially leads to postoperative neurological or vascular complications, which necessitate reoperations in 1 to 5% of all patients. In the established paradigm, a pedicle probe is inserted manually into the vertebra to create a pilot hole, a trajectory that the screw follows. It is difficult to achieve a stable, or even safe, trajectory with the limited physical feedback from the probe, so this technique is most often performed under fluoroscopic (X-ray) guidance. On average, spine surgeons take about 8-14 fluoroscopic shots per screw, resulting in radiation exposure 10-12 times greater than other musculoskeletal procedures.

Accordingly, there is a need in the art for a pedicle probe for the accurate placement of pedicle screws in spinal fusion surgeries, which also reduces screw breach rates, operating room time and radiation exposure, thereby, reducing the cost of the procedure, while enhancing safety for both patients and doctors.

### SUMMARY OF THE INVENTION

The foregoing needs are met, to a great extent, by the present invention which provides a device for spinal surgery including a shaft having a first end and a second end and an elongate length therebetween, wherein the first end is configured for probing a vertebra. The device includes a handle having a housing defining an interior space, wherein the second end of the shaft is configured to sit within at least a portion of the interior space. The device also includes a rotor configured to couple the shaft to the housing, and a force transducer configured to transmit data regarding forces sensed by the first end of the shaft. Additionally, the device includes a microprocessor configured to receive input from the force transducer and output data related to the forces sensed by the first end of the shaft, and a feedback system configured to transmit information to a user.

In accordance with an aspect of the present invention, the microprocessor is loaded with a non-transitory computer readable medium programmed to determine when a cortical wall of the vertebra is breached. The microprocessor can also be programmed to receive force readings from the force transducer. The microprocessor is programmed to calculate a moving average statistical technique for continuous point-to-point comparisons of the force readings. The microprocessor is also configured to activate the feedback system when detected forces exceed the pre-programmed threshold.

In accordance with yet another aspect of the present invention, the feedback system uses vibration, and the source of vibration can take the form of a vibrational motor. The feedback system can also take the form of lights, which can be LED lights. The feedback system can also include a combination of vibration and lights. The force transducer takes the form of torque sensing force transducers. Further, the device includes an internal power source.

In accordance with still another aspect of the present invention, the device includes a means of dynamically modulating the signal gain on the output of the force transducers, dependent on the amplitude of the input pressure. The device includes a means of continuously measuring a depth of penetration of a probe tip into a vertebra during a procedure. The means of continuously measuring a depth of penetration relays the data to the microprocessor as an input. The measurement is accomplished using electrical components. The measurement, alternately, is accomplished using mechanical components. The device also includes an inertial sensor configured to continuously measure the orientation of the probe relative to a fixed plane, which is relayed as an input to the microprocessor.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a side view of a spinal surgical probe, according to an embodiment of the present invention.

FIG. 2 illustrates an exploded view of a spinal surgical probe, according to an embodiment of the present invention.

FIG. 3 illustrates a partially exposed perspective view of a spinal surgical probe, according to an embodiment of the present invention.

FIG. 4A illustrates a partially exposed, partially exploded view and FIG. 4B illustrates an enlarged perspective view of a spinal surgical probe, according to an embodiment of the present invention.

FIG. 5 illustrates a partially exposed perspective view of a spinal surgical probe, according to an embodiment of the present invention.

FIG. 6 illustrates a partially exposed perspective view of a spinal surgical probe, according to an embodiment of the present invention.

FIGS. 7A and 7B illustrate perspective views of a shaft of the probe, according to an embodiment of the present invention.

FIG. 8 illustrates a partially exposed perspective view of a spinal surgical probe, according to an embodiment of the present invention.

FIG. 9 illustrates a partially exposed perspective view of a spinal surgical probe, according to an embodiment of the present invention.

FIG. 10 illustrates a partially exposed, partially exploded perspective view of a spinal surgical probe, according to an embodiment of the present invention.

FIG. 11 illustrates a partially exposed, partially exploded perspective view of a spinal surgical probe, according to an embodiment of the present invention.

FIG. 12 illustrates a partially exposed perspective view of a spinal surgical probe, according to an embodiment of the present invention.

FIG. 13 illustrates a flow diagram of a method according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout.

The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to an innovative pedicle probe that uses a force-sensing electromechanical system coupled with haptic and visual feedback. The probe of the present invention reduces the rate of pedicle screw breaches during spinal fusion surgery. The probe provides an effective guidance system to aid surgeons in detecting and preventing cortical bone breaches, thereby minimizing risk of intraoperative injury to the patient. Moreover, the probe invention decreases surgeon reliance on intraoperative radiation, reducing harmful exposure to both patients and surgeons.

The present invention relies on an electromechanical system to detect breaches in the cortical wall of the vertebrae. A combination of three force transducers is placed around the linear and rotational axis of the probe shaft. Two transducers measure bi-directional torque, and one is used to measure unidirectional pushing linear force. The two torque transducers are placed on the contact surfaces between a rotor and the probe body. The linear transducer is placed on the contact surface at the proximal end of the shaft. While two torque transducers and one linear transducer are disclosed herein, any suitable number of transducers known to one of skill in the art could be used. The shaft is not fixed to the probe handle but instead to the rotor; thus, it has a limited range over which it can displace along rotational and linear axes in order to act on the force transducers.

A microprocessor, which can take the form of an Arduino microprocessor, is used to process both outputs from the transducers. The microprocessor is loaded with a non-transitory computer readable medium, programmed with a novel moving average statistical algorithm that is used to calculate breach of the cortical wall. When the cortical breach is detected, the device provides visual and tactile feedback using a combination of vibrational motors and LED lights placed in the probe handle. The entirety of the electromechanical system is housed together in the handle of the probe, and powered by an internal power source. The internal power source can be rechargeable or replaceable batteries, a wired power connection or any other suitable means of powering the device known to or conceivable by one of skill in the art. While a handle design is disclosed herein, this particular design is not meant to be considered limiting, and this system may be housed in a variety of handle designs known to or conceivable by one of skill in the art. The present invention requires no additional input from the user and is entirely self-contained.

The scientific principle behind the present invention is based on density differences in the vertebrae. In studies of human vertebral anatomy, a significant density differential between higher-density cortical bone surrounding the vertebrae and lower-density cancellous bone within the vertebrae was identified. The present invention significantly reduces the risk of breaches of the cortical bone by providing real-time feedback to the surgeon to ensure that the probe is positioned within the lower-density bone. This is accomplished using an electromechanical method to continuously measure changes in force, when navigating the probe inside bone. The probe device measures torque and linear pushing forces with respect to the shaft, allowing for a holistic evaluation of the forces applied during the surgery. By coupling the force profile with a path-identification algorithm, the present invention alerts the surgeon to a potential or impending breach, all in real time, preventing the probe from exiting the vertebral body.

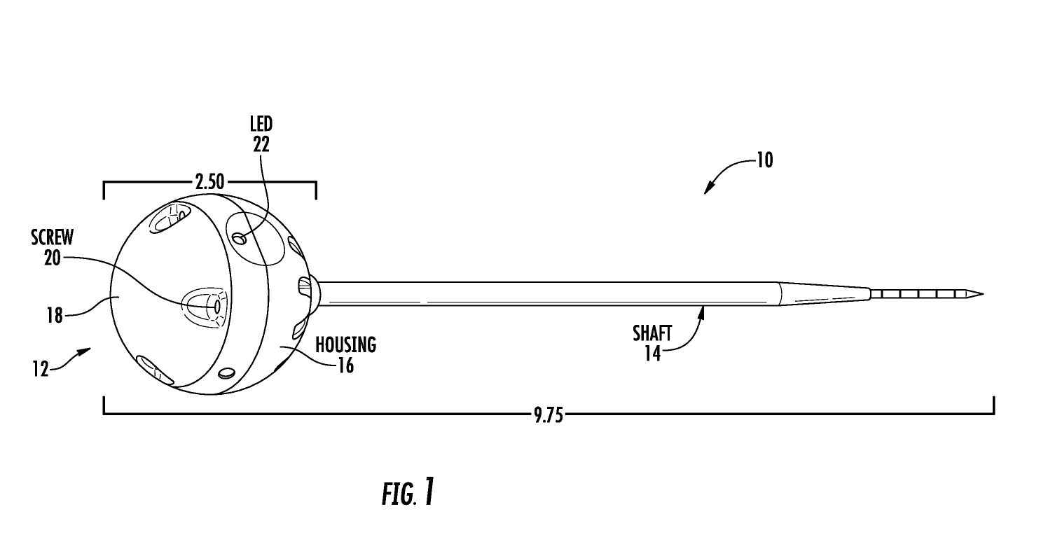

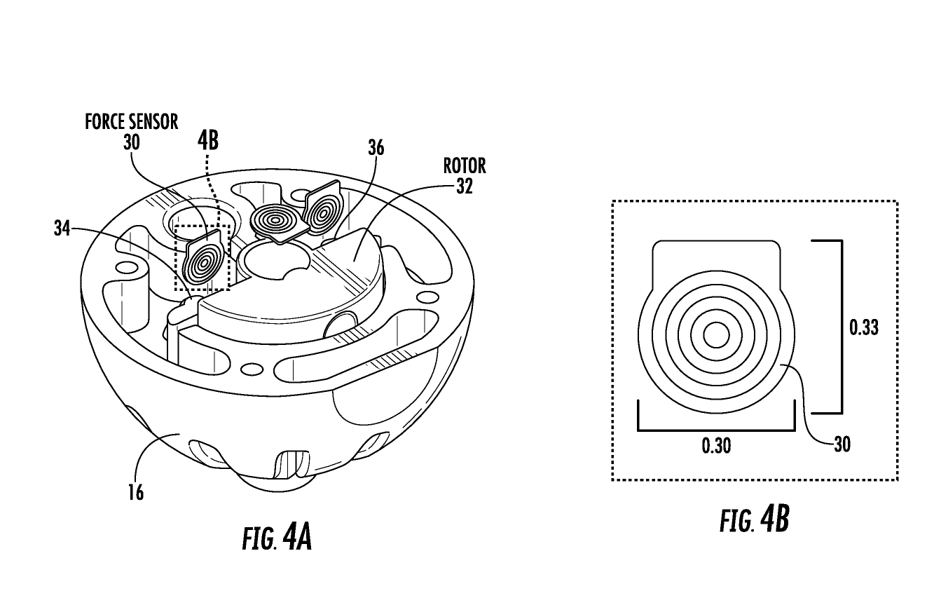

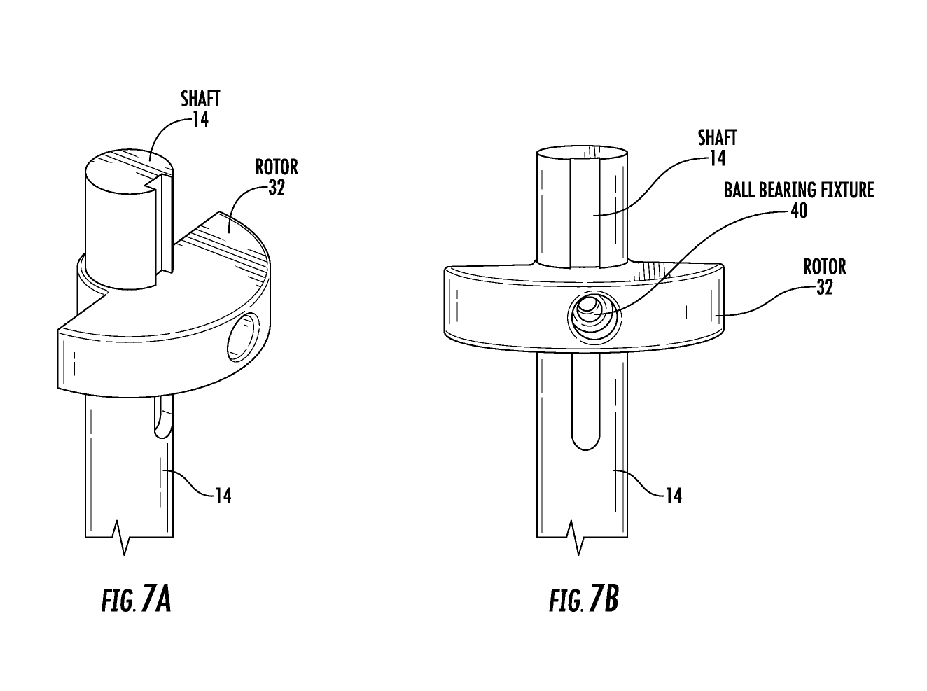

The present invention is a self-contained solution, which transforms spinal fixation, enabling safe and accurate pedicle screw placement across a broad range of patients. At the core of the present invention is an electromechanical system that measures the different forces and torques being applied to the probe over the course of an operation. The probe of the present invention includes a shaft and a handle. The shaft is connected to a semi-circular rotor by means of an opening and a fixative element such as a ball bearing fixture or set screw, as illustrated in FIGS. 7A and 7B. As illustrated in FIGS. 1, 2, 3, 4A and 4B, and 5, the present invention includes a handle defining a spherical housing. While shown as a spherical housing the handle can take any suitable shape known to or conceivable by one of skill in the art. The flat surfaces of the rotor orthogonal to the axis of the shaft present against flat faces within the spherical housing of the handle, as illustrated in FIGS. 1, 2, 3, 4A and 4B, and 5. Torque-sensing force transducers are placed in the space defined between these flat surfaces, as illustrated in FIGS. 1, 2, 4A and 4B, and 6. Thus, the motion of the surgeon in torqueing the probe head (the spherical housing) is sensed by compression against these sensors. Two lubricant-impregnated bushings are used to fix the orientation of the shaft; one is located on each side of the rotor. These bushings are fixed within the housing, but allow free rotation of the shaft. The proximal end of the shaft (the terminus closer to the user) is a flat surface facing a flat surface located within the housing. Between these surfaces is the force transducer used to sense linear force. The housing has a small tolerance for vertical displacement of the shaft (along its axis) such that pushing the probe results in compression against this sensor, as illustrated in FIGS. 4A and 4B, 5, and 6.

More particularly, FIG. 1 illustrates a side view of a spinal surgical probe, according to an embodiment of the present invention. The spinal surgical probe 10 includes a handle housing 12 and a shaft 14. The handle housing 12 is positioned proximal to the shaft 14. The housing 14 can include a two-piece construction having a distal portion 16 and a proximal portion 18. The two portions 16 and 18 of the housing 12 can be held together with screw 20. As will be illustrated further herein, the housing 12 can define an interior portion that is revealed and accessible by separating the two portions 16 and 18. The housing 12 can also include an LED 22 that is used to indicate cortical breach during screw placement.

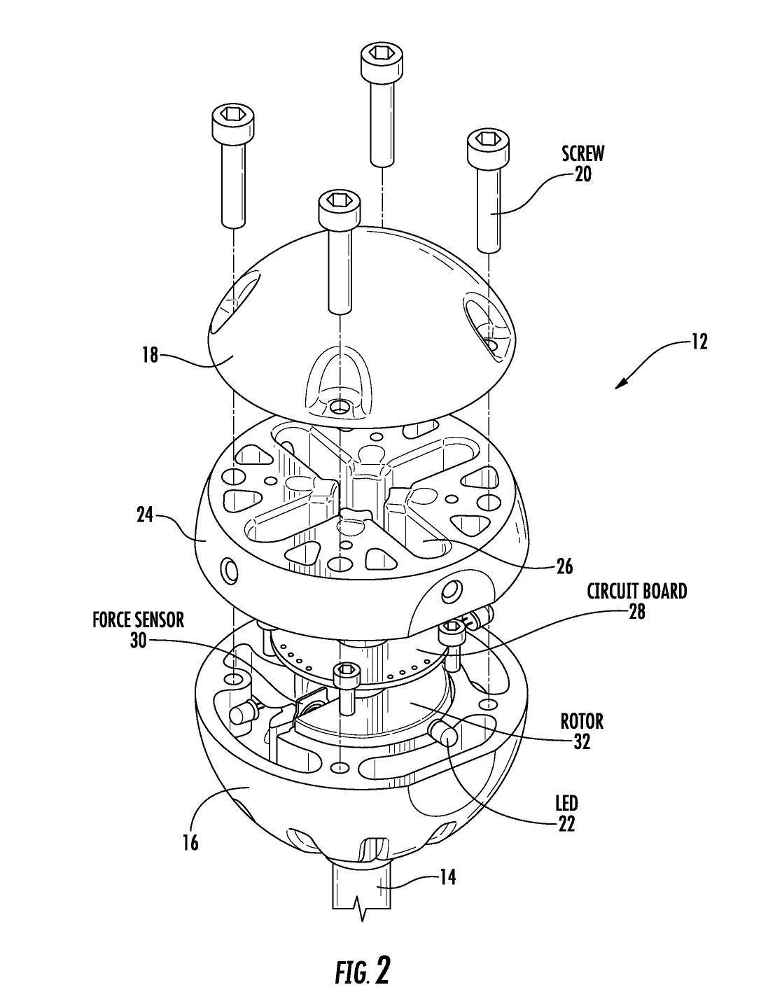

FIG. 2 illustrates an exploded view of a housing of a spinal surgical probe, according to an embodiment of the present invention. As illustrated in FIG. 2, the housing 12 includes distal portion 16 and proximal portion 18. The housing 12 can also include a middle portion 24 disposed between the distal and proximal portions 16 and 18. The middle portion can provide additional support for the components disposed within interior space 26 of the housing 12. Screws 20 hold the distal, middle, and proximal portions 16, 18, and 24 together. FIG. 2 illustrates a circuit board 28, force sensor 30, and rotor 32 disposed within the interior space 26 of the housing 12. These components are used to detect breach of cortical bone and are in communication with one another and LED 22. The rotor is coupled to the shaft 14 in order to limit rotational motion of the shaft. Therefore, the shaft has a limited range over which it can displace along rotational and linear axes in order to act on the force sensor 30.

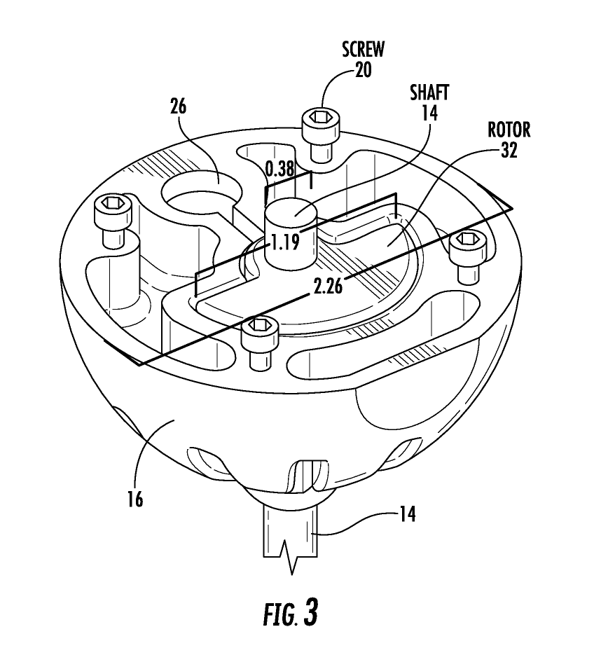

FIG. 3 illustrates a partially exposed perspective view of a spinal surgical probe, according to an embodiment of the present invention. FIG. 3 further illustrates the internal components of the surgical probe. Shaft 14 extends through the proximal portion 16 into the interior space 26 of the housing 12. The shaft 14 is coupled to rotor 32. The rotor 32 limits rotational motion of shaft 14.

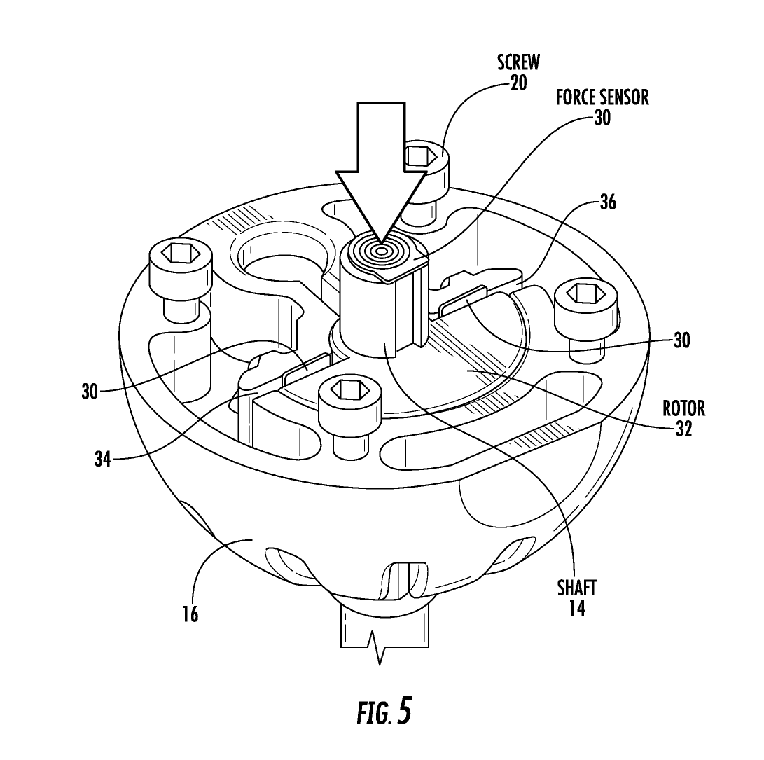

FIG. 4A illustrates a partially exposed, partially exploded view and FIG. 4B illustrates an enlarged perspective view of a spinal surgical probe, according to an embodiment of the present invention. As illustrated in FIG. 4A the distal portion of the housing 16 supports the rotor 32 and also defines spaces 34, 36 in which the force sensors 30 can be disposed. One force sensor 30 is disposed atop the rotor 32 in parallel with a top surface of the rotor 32, while the other two force sensors 30 are disposed in space defined by the proximal portion 16 of the housing substantially perpendicular to a side surface of the rotor 32. FIG. 4B illustrates an expanded view of one of the force sensors 30. FIG. 5 illustrates a partially exposed perspective view of a spinal surgical probe, according to an embodiment of the present invention. FIG. 5 further illustrates the spinal surgical probe of FIG. 4A with all of the force sensors 30 disposed in their placements. One force sensor 30 is disposed in space 34, one in space 36, and one on top of shaft 14. While an exemplary placement of force sensors is provided herein, this is not meant to be considered limiting, and any suitable arrangement of force sensors known to or conceivable by one of skill in the art could be used.

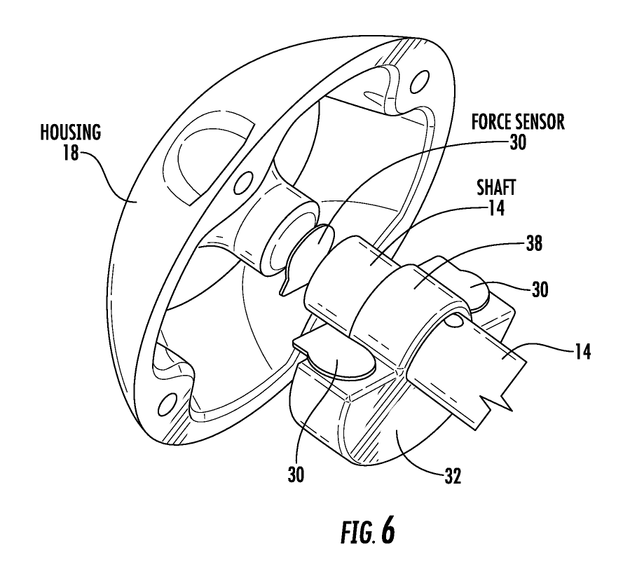

FIG. 6 illustrates a partially exposed perspective view of a spinal surgical probe, according to an embodiment of the present invention. As, illustrated in FIG. 6 the rotor 32 is coupled to the shaft 14 with shaft-rotor connector piece 38 disposed about the shaft 14. Force sensors 30 are disposed about the surface of the rotor 32 and the shaft 14. One force sensor 30 is disposed between the shaft 14 and the proximal portion 18 of the housing. The other two force sensors 30 are disposed on the vertical faces of the rotor 32 (rotor faces are vertical when the device is in a vertical position.

FIGS. 7A and 7B illustrate perspective views of a shaft of the probe, according to an embodiment of the present invention. As illustrated in FIGS. 7A and 7B, the rotor 32 is disposed at least partially surrounding shaft 14. The rotor 32 includes shaft-rotor connector piece 38 to ensure that the rotor 32 and the shaft 14 are coupled. The rotor 32 can also include a ball bearing assembly 40. The shaft 14 is connected to the generally semi-circular rotor 32 by means of an opening and a fixative element such as a ball bearing fixture 40 or set screw. Any other means of fixation known to or conceivable to one of skill in the art could also be sued.

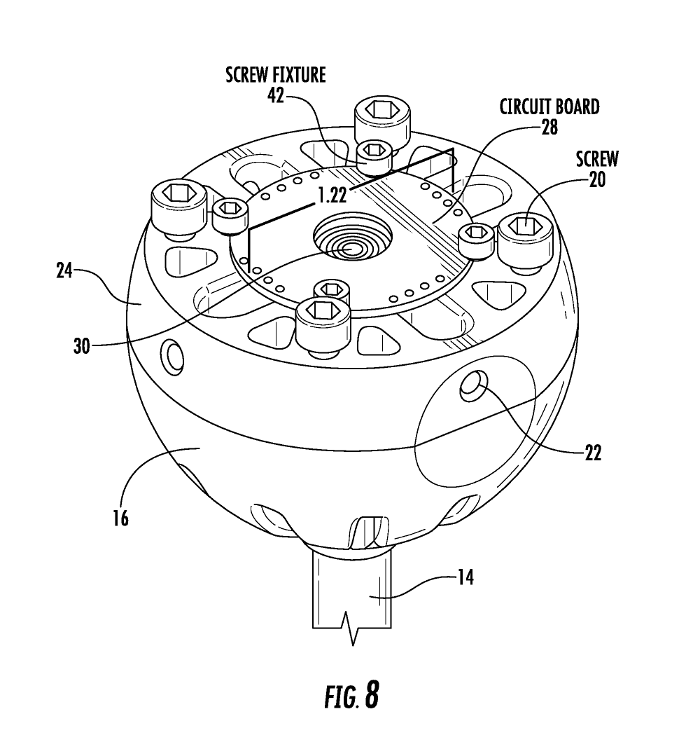

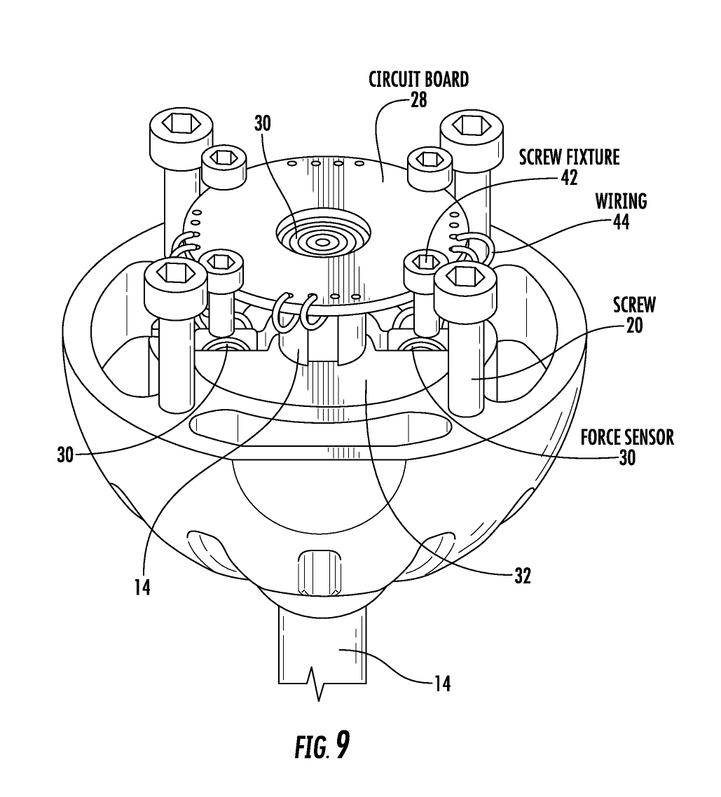

An electronics suite is also integrated within the spherical housing. This suite consists of a printed circuit board (PCB with an integrated microprocessor, illustrated in FIGS. 8, 9, and 10). The output wires of all three force transducers are connected to the PCB so that data is continuously provided to the microprocessor for analysis, as illustrated in FIG. 9.

FIG. 8 illustrates a partially exposed perspective view of a spinal surgical probe, according to an embodiment of the present invention, and FIG. 9 illustrates a partially exposed perspective view of a spinal surgical probe, according to an embodiment of the present invention. As illustrated in FIGS. 8 and 9, the circuit board 28 is disposed atop a surface of the middle portion 24 of the housing. The circuit board 28 is held in place with screw fixtures 42. Screws 20 are used to hold the middle portion 24 and the distal portion 16 together. Shaft 14 is disposed through the distal housing portion 16 and the middle housing portion 24 and is topped with a force sensor 32. The circuit board 28 is connected to LEDs 22 with wiring 44. The wiring can also be used to couple the circuit board to other components within the device, such as the force sensors.

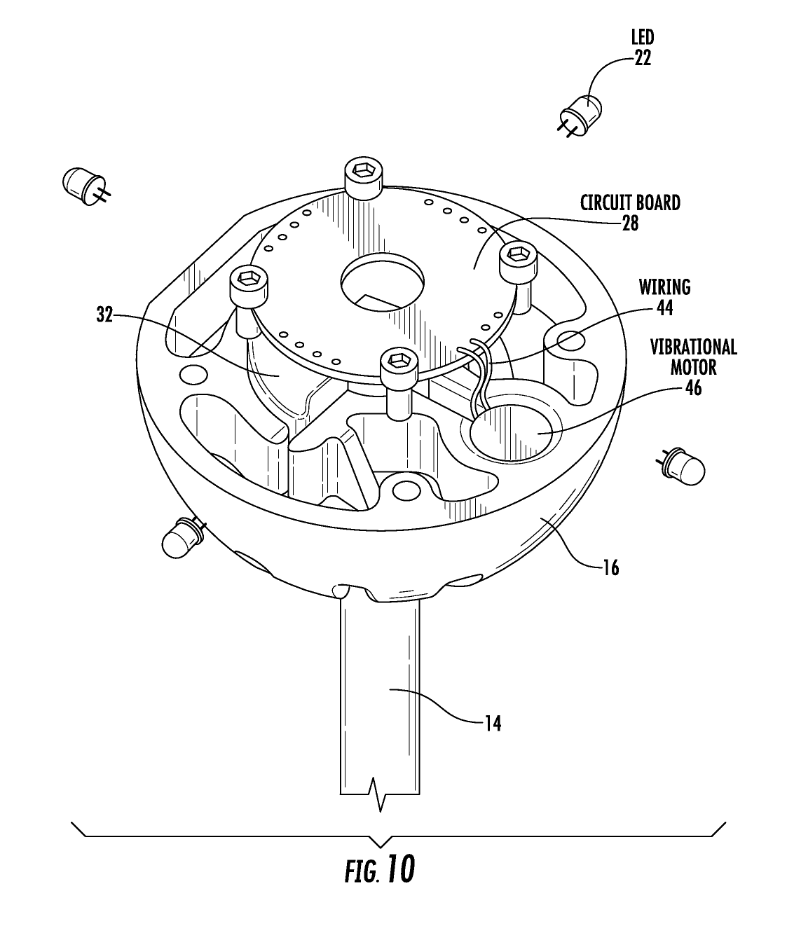

FIG. 10 illustrates a partially exposed, partially exploded perspective view of a spinal surgical probe, according to an embodiment of the present invention. As illustrated in FIG. 10, wiring 44 is also used to couple the circuit board 28 to vibrational motor 46. The vibrational motor 46 is disposed in a space defined by the proximal portion 16 of the housing. It provides feedback to the physician related to cortical breach.

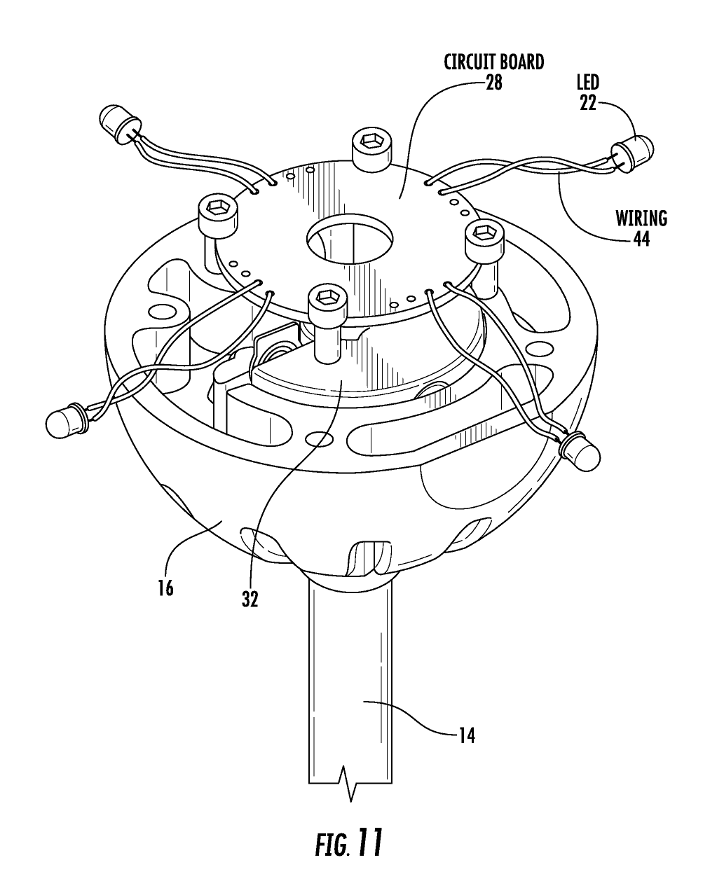

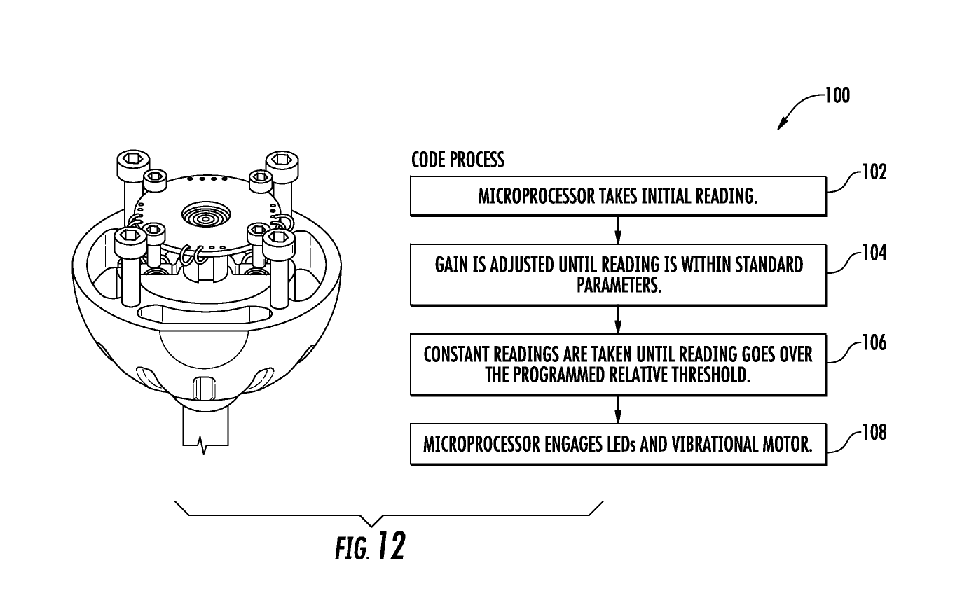

A vibrational motor housed within the proximal portion 16 of the handle 12 is wired to the circuit board 28 with wiring 44 to provide haptic feedback, as illustrated in FIGS. 10 and 11. Additionally, an array of LED lights 22 nested along the surface of the proximal portion 16 of the handle is wired to the circuit board 28 with wiring 44 to provide visual feedback, as illustrated in FIG. 11. These alert systems are controlled by the microprocessor such that when it is determined that a breach is imminent, the vibrational motor and LED are activated, as illustrated in FIG. 12.

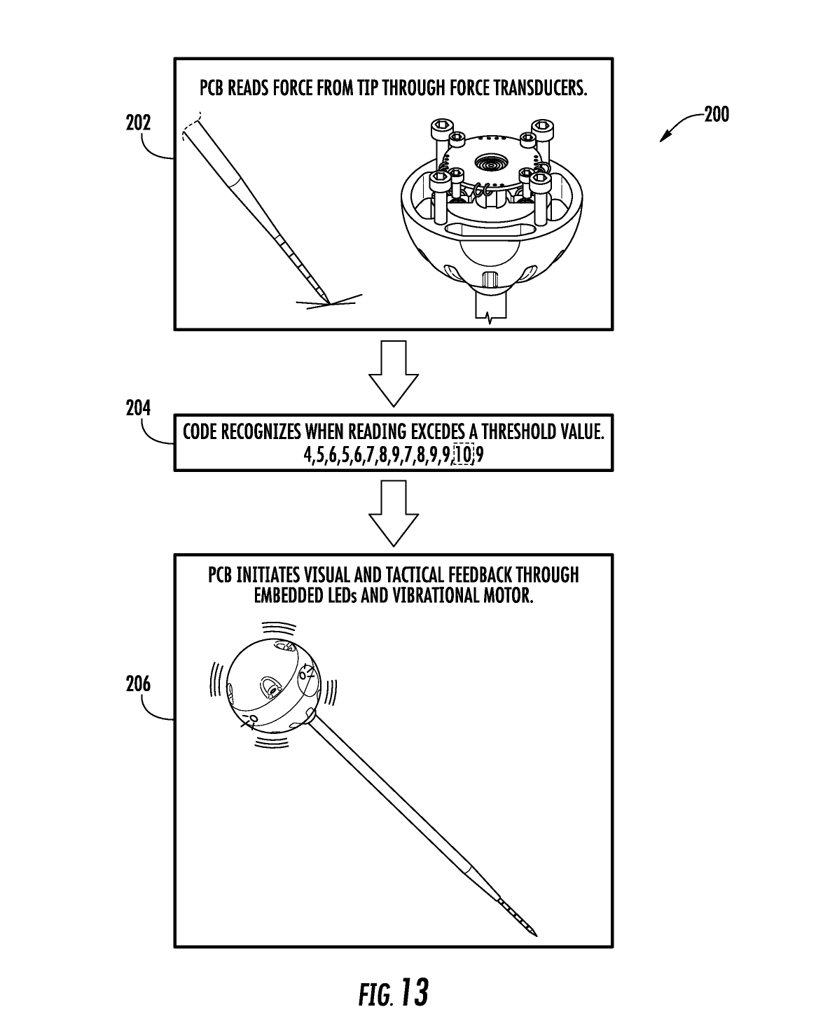

The microprocessor is programmed with a non-transitory computer readable medium programmed with proprietary code written in C with an open source library. In the course of its use, force readings are taken at 10 kHz by the force transducers placed along the rotational and linear axis. The discrimination is achieved using a moving average statistical technique for continuous point-to-point comparisons of the output from the transducers, as illustrated in FIGS. 12 and 13. When the profile of detected forces exceeds the pre-programmed threshold, the feedback mechanisms are activated, as illustrated in FIG. 13.

FIG. 12 illustrates a flow diagram for a code process, according to an embodiment of the present invention. As illustrated in FIG. 12 the code process 100 includes a step 102 of the microprocessor taking an initial reading from the force sensors. Step 104 includes adjusting gain until a reading is within a standard parameter. Step 106 includes taking constant readings until the reading is over the programmed relative threshold for force from the force sensors. Step 108 includes the microprocessor engaging the LEDs and vibrational motor in order to warn the physician of impending cortical breach.

FIG. 13 illustrates a flow diagram of protocol when the force exceeds the pre-programmed threshold and the feedback mechanisms are activated. The flow diagram 200 shows step 202 of the circuit board reading force from the tip of the device via readings from the force sensors. The non-transitory computer readable medium is programmed to recognize when a reading exceeds a threshold value, in step 204. Step 206 includes the circuit board initiating visual and tactical feedback through embedded LEDs and a vibrational motor. In practice the physician turns the spinal screw with the device. The device uses the force sensors to determine the force at the tip of the device. These force sensor readings are analyzed by the program on the non-transitory computer readable medium, which looks for a force value that is greater than the preset force limit. Once this limit is exceeded haptic and or visual feedback is provided to the user to indicate impending cortical breach.

The probe of the present invention is intended to minimize complexity and fit within the established surgical paradigm. The device is used by inserting the probe shaft into the opening in the pedicle previously created by a surgical burr and pushing through the softer cancellous bone inside the vertebrae. This method of use of the device is identical to the standard of the contemporary pedicle probe. When it is determined that the shaft comes into contact with denser surrounding cortical bone, the surgeon is alerted by visual and haptic feedback. The vibrational motors are activated and the LED lights blink to alert the surgeon of the impending breach.

It should be noted that herein that any algorithms or methods of the present invention described above can be carried out using a microprocessor or a computer loaded with a non-transitory computer readable medium, independent of or incorporated with the system. Indeed, any suitable method of calculation known to or conceivable by one of skill in the art could be used. The computing device, microprocessor, or other means for calculating can be designed specifically for the present invention, such that it is small enough to fit within the housing and is configured to communicate wirelessly or via a direct electrical connection, among other design and electrical considerations.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. These methods could also be executed by a computer application loaded on a smartphone, PC, tablet, phablet or other computing device that receives the data via the BlueTooth connection disposed within the circuit.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A device for spinal surgery comprising:
a shaft having a first end and a second end and an elongate length therebetween, wherein the first end is configured for probing a vertebra;
a handle having a housing defining an interior space, wherein the second end of the shaft is configured to sit within at least a portion of the interior space;
a rotor configured to couple the shaft to the housing;
a force transducer configured to transmit data regarding forces sensed by the first end of the shaft;
a microprocessor configured to receive input from the force transducer and output data related to the forces sensed by the first end of the shaft; and
a feedback system configured to transmit information to a user.

2. The device of claim 1 further comprising a microprocessor loaded with comprising a non-transitory computer readable medium programmed to determine when a cortical wall of the vertebra is breached.

3. The device of claim 1 further comprising a microprocessor loaded with comprising a non-transitory computer readable medium programmed to use the penetration depth, force data, and orientation data as inputs to determine position of the device; to compute a likelihood of cortical breach, to activate the feedback system when a pre-programmed threshold is reached.

4. The device of claim 3 further comprising the microprocessor being configured for using pre-operative scan data to calibrate position of the probe relative to a patient or vertebra.

5. The device of claim 3 further comprising the microprocessor being configured for outputting calculated probe position data overlaying a scan of a vertebra of a patient.

6. The device of claim 5 further comprising the microprocessor being configured for outputting the data with a wired transmission over a cable extending from the handle.

7. The device of claim 5 further comprising the microprocessor being configured for outputting the data with a wireless transmission from a transmitter positioned within an interior space of the handle to a receiver external to the probe.

8. The device of claim 1 wherein the feedback system comprises vibration.

9. The device of claim 1 wherein the feedback system comprises a vibrational motor.

10. The device of claim 1 wherein the feedback system comprises lights.

11. The device of claim 10 wherein the feedback system comprises LEDs.

12. The device of claim 1 wherein the feedback system comprises vibration and lights.

13. The device of claim 1 wherein the force transducer comprises torque sensing force transducers.

14. The device of claim 1 further comprising an internal power source.

15. The device of claim 1 further comprising a means of dynamically modulating the signal gain on the output of the force transducers, dependent on the amplitude of the input pressure.

16. The device of claim 1 further comprising a means of continuously measuring a depth of penetration of a probe tip into a vertebra during a procedure.

17. The device of claim 16 further comprising the means of continuously measuring a depth of penetration relaying the data to the microprocessor as an input.

18. The device of claim 16 wherein the measurement is accomplished using electrical components.

19. The device of claim 16 wherein the measurement is accomplished using mechanical components.

20. The device of claim 1 further comprising an inertial sensor configured to continuously measure the orientation of the probe relative to a fixed plane, which is relayed as an input to the microprocessor.
